(19) United States
(12) Patent Application Publication
(10) Pub. No.: US 2022/0104920 A1
(43) Pub. Date: Apr. 7, 2022

(54) METHODS AND SYSTEMS FOR DESIGNING DENTAL APPPLIANCES

(71) Applicant: Align Technology, Inc., San Jose, CA (US)

(72) Inventors: Yuxiang WANG, San Jose, CA (US); Reza SHIRAZI AGHJARI, Campbell, CA (US); Luyao CAI, San Jose, CA (US); Rohit TANUGULA, San Jose, CA (US); Jun SATO, San Jose, CA (US); Vadim MATOV, San Jose, CA (US); Bastien PESENTI, San Jose, CA (US); Manlio VALDIVIESO, San Jose, CA (US); Peter WEBBER, San Mateo, CA (US); Mikhail Nikolaevich RYCHAGOV, San Jose, CA (US)

(21) Appl. No.: 17/449,799

(22) Filed: Oct. 1, 2021

Related U.S. Application Data
(60) Provisional application No. 63/087,163, filed on Oct. 2, 2020.

(51) Int. Cl. A61C 7/00; A61C 7/08; G16H 30/40

(52) U.S. Cl. CPC A61C 7/002; G16H 30/40; A61C 7/08

(57) ABSTRACT

Computer-implemented methods and systems for designing oral appliances. These methods and systems may include receiving patient data and a treatment plan (including a proposed one or more dental appliances), and determining, using the patient data and the treatment plan, an improved appliance design for treating the patient. These methods and systems may include evaluating the appliance design based on simulation and finalizing the appliance design based on the evaluation.

### CLAIM OF PRIORITY

This patent application claims priority to U.S. Provisional Patent Application No. 63/087,163, titled “SIMULATED EFFICACY,” and filed on Oct. 2, 2020, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Orthodontic treatment planning involves various steps before an appropriate oral appliance is fabricated for the patient. Patient records may be collected. For example, data on the patient's mouth may be scanned optically or obtained via polyvinyl siloxane impressions. Once the patient's mouth is digitized, a practitioner may plan out final teeth positions to treat the patient. Although software tools may aid during the design process, such tools may rely on certain assumptions. For example, the software tools may assume that the patient's teeth will move exactly as planned. Additionally, the software tools may rely on generalized clinical data. However, these assumptions may not account for the various biomechanical interactions that occurs in each patient's mouth. Moreover, complex cases may benefit from additional scanning to improve the treatment plan. The practitioner may not initially be able to determine when to prescribe additional scanning.

### SUMMARY OF THE DISCLOSURE

Described herein are systems and methods for improving treatment planning and orthodontic appliance design. In particular, described herein are methods and systems that simulate the action of a proposed treatment plan, such as one or more dental appliances (e.g., dental aligners) to execute the treatment plan, on a patient's teeth and comparing the resulted simulated tooth positions to a desired tooth position based on the treatment plan. The simulated tooth positions may include a simulation of bone remodeling. The simulation may be based on patient-specific information (e.g., patient anatomy, one or more scans, such as CBCT data, etc.). The simulation and resulting comparison may then be used to modify the design of one or more dental appliances (e.g., aligners).

The systems and methods described herein may improve the functioning of a computing device by reducing computing resources and overhead for simulating treatment results, thereby improving processing efficiency of the computing device over conventional approaches. These systems and methods may also improve the field of orthodontic treatment by analyzing data to efficiently model teeth movement to optimize orthodontic appliance design. In particular, it may be advantageous to perform the simulation(s), including incorporating bone remodeling and/or periodontal ligament effects on treatment after the initial treatment plan has been generated, including generating an initial one or more dental appliances based on the treatment plan. For example the initial treatment plan and dental appliance design may be generated without the requirement of modeling the often complex behavior associated with bone remodeling and/or periodontal ligament. This may provide a surprising improvement in time efficiency and may allow the application of prior clinical data into the modeling of the bone remodeling and properties of the periodontal ligament(s). As a result, the methods and systems described herein may be significantly faster and may provide dramatic improvements in efficacy of the aligner.

The methods and systems described herein generally include remodeling of bone due to treatment in order to analyze and improve aligner design. Bone remodeling may be based on a variety of complex physiological properties. Although in general, the bone forming and/or supporting teeth may remodel if the force of movement on the bone exceeds a threshold valve (which may be based on one or more physiological properties, including type of tooth, shape of tooth, etc.), it has proven difficult to estimate what this threshold may be, and also how to apply such bone remodeling when treating patients. The methods and systems described herein illustrate mechanisms for modeling and applying bone remodeling. In some examples these methods may model both a ligament layer and a bone remodeling layer as part of the overall modeling and analysis.

The methods and systems described herein may also generally compare the desired position of the teeth during (and in particular at the end of) an orthodontic treatment based on a treatment plan. The comparison may be used to determine if a proposed treatment plan is adequate for achieving a desired outcome. Alternatively or additionally, the comparison may be used to modify the treatment plan and/or the one or more appliances implementing the treatment plan. For example, if the difference between the proposed (desired) tooth position of one or more teeth is different from the simulated position (where the simulated position accounts for bone remodeling as described herein) is greater than a threshold (e.g., an “efficacy threshold”), the treatment plan and/or one or more dental appliances may be modified in order to reduce the difference between the desired and actual positions. For example, the method or system may modify the one or more dental appliances (e.g., aligners) to increase the forces acting on the teeth, e.g., by configuring the dental appliance(s) to overshoot the tooth position(s) in one or more appliances so that the desired tooth position is achieved.

These methods and systems may be used in a variety of different contexts. For example, these methods and systems may be used as tool for research and development. Any of these methods and system may be used to train tooth movement models for individuals, and/or may test solutions or components (e.g., attachments, aligner features, aligner track activations, elastics, etc.) when designing. These methods and systems may also be used with categories of patients, since patient's may be categorized into groups based on demographics, morphology etc. and solution tailored for them may be based on these categories. These methods and system may be used for commercial (e.g., production) to for treatment planning and/or manufacture of dental appliances, such as dental aligners (e.g., “shell aligners”).

Described herein are methods. These methods may be for designing an appliance for orthodontic treatment, and/or for forming a treatment plan. Any of these methods may include: receiving patient data and a treatment plan; determining, using the patient data and the treatment plan, an appliance design for treating the patient; evaluating the appliance design based on a bone remodeling simulation; and finalizing the appliance design based on the evaluation.

For example described herein are methods for designing an appliance for orthodontic treatment that include: receiving patient data, and a treatment plan comprising one or more appliances designed for treating a patient; simulating bone remodeling from the patient data to determine a simulated tooth position using the one or more appliances; evaluating the one or more appliances by comparing the simulated tooth position to the treatment plan; and modifying the one or more appliances when the comparison between the simulated tooth position and a desired tooth position from the treatment plan exceeds an efficacy threshold.

Thus, in any of these methods the bone remodeling simulation may use the appliance design to simulate an efficacy of the appliance design. Evaluating the appliance design may include redesigning the appliance design if the simulated efficacy does not satisfy an efficacy threshold. The efficacy threshold may correspond to a simulated final position compared to a desired final position and an efficacy value for at least one tooth. For example, the efficacy threshold may be, e.g., 75% (e.g., 76%, 77%, 78%, 79%, 80%, 81%, 82%, 83%, 84%, 85%, 86%, 87%, 88%, 89%, 90%, 91%, 92%, 93%, 94%, 95%, etc.). In some examples the efficacy threshold is 85%.

The simulation (e.g., the bone remodeling simulation, which may simulate tooth movement/position) may include using a 6 degree-of-freedom (DOF) spring to represent a periodontal ligament (PDL) and the bone remodeling is simulated to occur when the 6 DOF spring is displaced according to a force applied to the 6 DOF spring meeting a force criterion. The force criteria may correspond to a plastic flow rule. In some examples the force criteria correspond(s) to a nonlinear hardening rule. Any of these methods may include adjusting spring parameters of the 6 DOF spring based on the patient data. The methods may include adjusting spring parameters of the 6 DOF spring based on a tooth type corresponding to a periodontal ligament (PDL).

In any of these methods, the simulation may include a representation of the action of periodontal ligaments on the bone. For example, any of these methods may include, as part of the simulation(s)/model(s), a first array of springs distributed along a shape of bone to represent a periodontal ligament (PDL) and a second array of springs to represent bone remodeling. In general, the shape of bone is determined from imaging data. The bone remodeling may be based on elastic deformation. The bone remodeling may be based on a non-linear elastic model.

The simulation may include a geometry of bone and a geometry of periodontal ligament (PDL), and the bone remodeling simulation calculates stresses at a boundary between the geometry of bone and the geometry of PDL. The bone remodeling simulation may include a solid element that changes a density or a stiffness. The bone remodeling simulation may include a high viscosity fluid that flows when a strain criterion is satisfied. In any of these examples, the bone remodeling simulation may include a solid element that changes size based on a stress state of the solid element.

The bone remodeling simulation may include a bi-phasic element that shifts phases under a stress state.

In any of these methods the evaluation of the appliance design may also include evaluating an index of predictability and determining a treatment complexity based on the index of predictability. For example, the simulation may include contact modeling when the treatment complexity is low.

In general, evaluating the appliance design may further comprise: creating, using triangulation, a plurality of meshes corresponding to each tooth, attachment, and aligner; applying boundary conditions and mechanical properties to the plurality of meshes; determining contact points between the plurality of meshes; defining a displacement field, a normal contact force, and a shear contact force for each contact point; associating contact points that correspond to each tooth; for each tooth, determining which mesh nodes correspond to the associated contact points; for each current tooth position, determining a net contact force and force points for each associated contact point; and predicting a next tooth position for each current tooth position using the net contact force and force points. Applying boundary conditions and mechanical properties may be based on finite element modeling (FEM). Determining contact points may be based on finite element analysis (FEA). The simulation may include biomechanical modeling when the treatment complexity is high.

In general, the methods (e.g., the simulations) described herein may use cone beam computed tomography (CBCT) data.

In any of these examples, evaluating the appliance design may further comprise reconstructing three-dimensional (3D) shapes of at least one of: teeth, apex positions, teeth root collisions, and level of orthodontic security. The reconstruction may utilize a volumetric neural network. In some examples, the volumetric neural network includes at least one of a u-net and a v-net.

Also described herein are systems for performing any of the methods described herein. For example, described herein are systems comprising: one or more processors; and a memory coupled to the one or more processors, the memory configured to store computer-program instructions, that, when executed by the one or more processors, perform a computer-implemented method comprising: receiving patient data, and a treatment plan comprising one or more appliances designed for treating a patient; simulating bone remodeling from the patient data to determine a simulated tooth position using the one or more appliances; evaluating the one or more appliances by comparing the simulated tooth position to the treatment plan; and modifying the one or more appliances when the comparison between the simulated tooth position and a desired tooth position from the treatment plan exceeds an efficacy threshold.

Also described herein are non-transitory computer-readable medium comprising one or more computer-executable instructions that, when executed by at least one processor of a computing device, cause the computing device to perform any of these methods. In general, any of these systems may include such non-transitory computer-readable media. For example, described herein are non-transitory computer-readable medium comprising one or more computer-executable instructions that, when executed by at least one processor of a computing device, cause the computing device to: receive patient data, and a treatment plan comprising one or more appliances designed for treating a patient; simulate bone remodeling from the patient data to determine a simulated tooth position using the one or more appliances; evaluate the one or more appliances by comparing the simulated tooth position to the treatment plan; and modify the one or more appliances when the comparison between the simulated tooth position and a desired tooth position from the treatment plan exceeds an efficacy threshold.

In any of the methods and systems described herein, the modeling of the tooth movement may be done based on patient-specific parameters, as will be described in greater detail below. In some cases the modeling may be streamlined or accelerated by using reduced-basis modeling methods. For example, many full simulations may be run and find the representative “modes” or “patterns” of the model behavior, and this information may be used to accelerate the algorithm. This may allow the system or method to run a reduced model for every patient. Patient's may be categorized based on patient information (such as gender, age, tooth shape, etc.) and reduced models may be used within similar categories. Alternatively or additionally, the modeling described herein may be accelerated by running a finite set of trained models (e.g., few thousand) and training a machine learning model as a surrogate model, as a reduced order model to be deployed in production time to optimize the solution.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative examples, and the accompanying drawings of which:

FIG. 1 shows a block diagram of an example system for simulating efficacy using mechanical simulations, in accordance with some examples.

FIG. 2 shows a flow diagram of designing and evaluating an appliance design, in accordance with some examples.

FIG. 3 shows a diagram of bone remodeling, in accordance with some examples.

FIGS. 4A-4B show examples of mechanical models for simulating bone remodeling, in accordance with some examples. FIG. 4C illustrates forces acting on the model.

FIGS. 5A-B show an example model for simulating bone remodeling, in accordance with some examples.

FIGS. 6A-B show another example model for simulating bone remodeling, in accordance with some examples.

FIG. 7 shows a flow diagram of contact modeling, in accordance with some examples.

FIG. 8 shows a flow diagram of an example method for simulating efficacy, in accordance with some examples.

FIG. 9 shows a flow diagram of an example method for optimizing appliance design, in accordance with some examples.

FIG. 10 shows a block diagram of an example computing system capable of implementing one or more examples described and/or illustrated herein, in accordance with some examples; and

FIG. 11 shows a block diagram of an example computing network capable of implementing one or more of the examples described and/or illustrated herein, in accordance with some examples.

### DETAILED DESCRIPTION

The following detailed description and provides a better understanding of the features and advantages of the inventions described in the present disclosure in accordance with the examples disclosed herein. Although the detailed description includes many specific examples, these are provided by way of example only and should not be construed as limiting the scope of the inventions disclosed herein.

A patient's orthodontic treatment plan may require usage of various oral appliances across various stages which may span months. Treatment planning may utilize software tools to plan the various stages and associated appliances. As the treatment proceeds, the patient's teeth movement may deviate from the planned teeth movement, which may necessitate updated treatment planning.

As will be described further below, the systems and methods provided in this disclosure may utilize simulation-based feedback to optimize appliance design. The systems and methods provided in this disclosure may improve the functioning of a computing device by producing more accurate designs without requiring significantly more data, which may further reduce storage requirements and network bandwidth. In addition, the systems and methods provided herein may improve the field of orthodontic care by improving the initial treatment plans. Moreover, the systems and methods provided herein may improve the field of biomechanical modeling by applying models of increasing complexity.

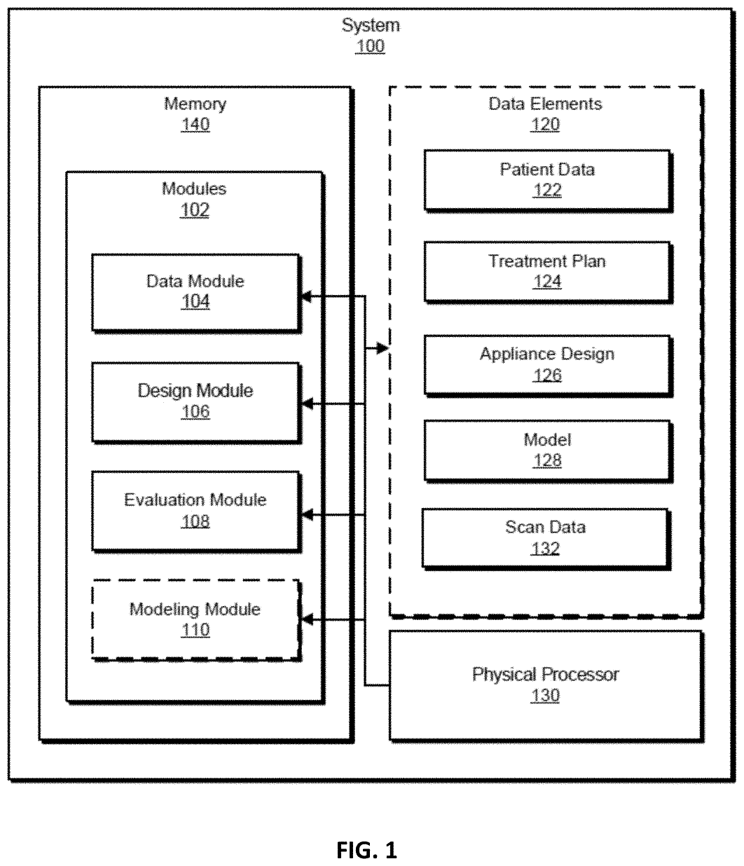

FIG. 1 is a block diagram of an example system 100 for using mechanical simulations to improve appliance design. System 100 generally represents any type or form of computing device that is capable of reading computer-executable instructions and storing and analyzing data. System 100 may be a backend server for running simulations using patient data and treatment data. Additional examples of system 100 include, without limitation, application servers, web servers, storage servers, and/or database servers configured to run certain software applications and/or provide various web, storage, and/or database services. Alternatively, system 100 may be, for example, a smartphone, an augmented reality device, or other consumer device. Additional examples of system 100 include, without limitation, laptops, tablets, desktops, servers, cellular phones, Personal Digital Assistants (PDAs), multimedia players, embedded systems, wearable devices (e.g., smart watches, smart glasses, etc.), smart vehicles, smart packaging (e.g., active or intelligent packaging), gaming consoles, so-called Internet-of-Things devices (e.g., smart appliances, etc.), variations or combinations of one or more of the same, and/or any other suitable computing device. Although illustrated as a single entity in FIG. 1, system 100 may include and/or represent a plurality of computing devices such as servers that work and/or operate in conjunction with one another.

As illustrated in FIG. 1, system 100 may include one or more memory devices, such as memory 140. Memory 140 generally represents any type or form of volatile or non-volatile storage device or medium capable of storing data and/or computer-readable instructions. In one example, memory 140 may store, load, and/or maintain one or more of modules 102. Examples of memory 140 include, without limitation, Random Access Memory (RAM), Read Only Memory (ROM), flash memory, Hard Disk Drives (HDDs), Solid-State Drives (SSDs), optical disk drives, caches, variations or combinations of one or more of the same, and/or any other suitable storage memory.

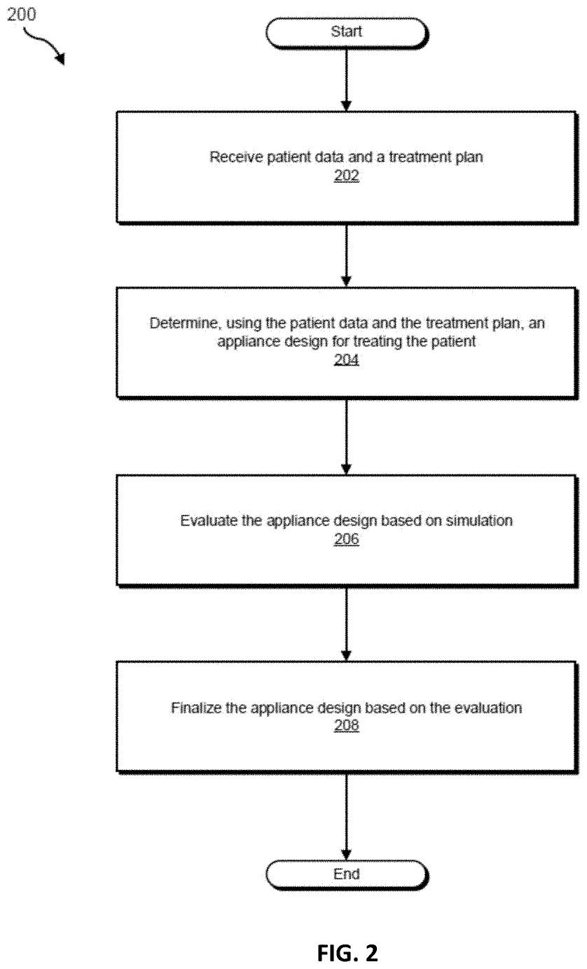

As illustrated in this figure, example system 100 may include one or more modules 102 for performing one or more tasks. As will be explained in greater detail below, modules 102 may include a data module 104, a design module 106, an evaluation module 108, and in some implementations a modeling module 110. Although illustrated as separate elements, one or more of modules 102 in FIG. 1 may represent portions of a single module or application. As will be described in greater detail below, one or more of modules 102 from FIG. 1 may, when executed by at least one processor of system 100, enable system 100 to simulate a treatment plan. For example, and as will be described in greater detail below, one or more of modules 102 may cause system 100 to perform the steps described in FIGS. 2, 7, 8, and 9.

In certain examples, one or more of modules 102 in FIG. 1 may represent one or more software applications or programs that, when executed by a computing device, may cause the computing device to perform one or more tasks. For example, and as will be described in greater detail below, one or more of modules 102 may represent modules stored and configured to run on one or more computing devices, such as system 100. One or more of modules 102 in FIG. 1 may also represent all or portions of one or more special-purpose computers configured to perform one or more tasks.

As illustrated in FIG. 1, system 100 may also include one or more physical processors, such as physical processor 130. Physical processor 130 generally represents any type or form of hardware-implemented processing unit capable of interpreting and/or executing computer-readable instructions. In one example, physical processor 130 may access and/or modify one or more of modules 102 stored in memory 140. Additionally or alternatively, physical processor 130 may execute one or more of modules 102 to facilitate running simulations to improve appliance designs. Examples of physical processor 130 include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, and/or any other suitable physical processor.

As illustrated in FIG. 1, example system 100 may also include one or more data elements 120, such as patient data 122, treatment plan 124, appliance design 126, model 128, and scan data 132. Data elements 120 generally represents any type or form of data or information. Patient data 122 may include any data relating to a patient, such as dentition data, patient scans (e.g., orthopantomogram, periapical, lateral cephalography), photographs, specific tooth data, patient information, patient demographics, notes regarding the patient, etc. Treatment plan 124 may include any data relating to a treatment for the patient, such as a specific treatment plan (e.g., various stages of a complete treatment plan), portions of a treatment plan, general guidelines for treatment, desired teeth positions, etc. Appliance design 126 may include any data relating to oral appliances, such as specific designs and models, fabrication techniques, etc. Model 128 may include any data relating to mechanical simulation models, such as bone remodeling models, parameters for models, simulation results, three-dimensional (3D) models, other simulation data, etc. Scan data 132 may include any data scanned from the patient, such as higher resolution scans of the patient, tomographic scans, cone beam computed tomography (CBCT) data, etc. Although illustrated as separate data elements, in certain implementations data elements 120 may be organized in another fashion and may include overlap between certain data elements 120.

FIG. 2 is a flow diagram of an exemplary computer-implemented method 200 for simulated efficacy. The steps shown in FIG. 2 may be performed by any suitable computer-executable code and/or computing system, including the system(s) illustrated in FIG. 1. In one example, each of the steps shown in FIG. 2 may represent an algorithm whose structure includes and/or is represented by multiple sub-steps, examples of which will be provided in greater detail below.

As illustrated in FIG. 2, at step 202 one or more of the systems described herein may receive patient data and a treatment plan. For example, data module 104 may receive patient data 122 and treatment plan 124.

The systems described herein may perform step 202 in a variety of ways. In one example, system 100 may receive patient data 122 and/or treatment plan 124 from another computing device. For example, system 100 may receive patient data 122 from a database storing patient data and may receive treatment plan 124 from a database storing treatment plans. In other examples, system 100 may directly collect patient data 122 and/or treatment plan 124. For instance, system 100 may directly scan the patient or otherwise have patient data 122 input into system 100. Likewise, treatment plan 124 may be directly input into system 100.

At step 204 one or more of the systems described herein may determine, using the patient data and treatment plan, an appliance design for treating the patient. For example, design module 106 may determine appliance design 126 using patient data 122 and treatment plan 124.

In some examples, the term “appliance design” may refer to one or more layouts, configurations, designs, instructions, etc. which may be used to fabricate an appliance. An appliance design may include various designs for multiple stages of treatment. Examples of appliance designs include, without limitation, 3D models, molds, configuration data, dimensions, etc.

The systems described herein may perform step 204 in a variety of ways. In one example, treatment plan 124 may be applied to patient data 122 to produce appliance design 126. In other examples, treatment plan 124 may be converted into appliance design 126.

At step 206 one or more of the systems described herein may evaluate the appliance design based on simulation. For example, evaluation module 108 may evaluate appliance design 126 based on a simulation using model 128.

The systems described herein may perform step 206 in a variety of ways. In one example, modeling module 110 may generate model 128 and further run a simulation using model 128. Modeling module 110 may use one or more machine learning or other artificial intelligence schemes.

In some examples, the simulation may include a bone remodeling simulation using appliance design 126 to simulate an efficacy of appliance design 126. For example, the simulation may include a bone remodeling simulation to simulate the efficacy of appliance design 126. Evaluation module 108 may compare the simulated efficacy against an efficacy threshold. The efficacy threshold may correspond to a simulated final position compared to a desired final position and an efficacy value for at least one tooth. For instance, the efficacy value for a tooth may correspond to how close the simulated final position of the tooth is to the desired final position of the tooth (e.g., based on distance, orientation, etc.) and may be numerically represented, such as a percentage. The efficacy threshold may be, for example, 85%, 80%, 90%, or any other appropriate threshold value. If the simulated efficacy does not satisfy the efficacy threshold, design module 106 may redesign appliance design 126.

Modeling module 110 may determine the simulated final position of a tooth by mechanically simulating movement of the tooth and surrounding parts. The simulation may use model 128 for initializing the simulation. In some examples, the mechanical simulation may include simulating bone remodeling of an examined tooth.

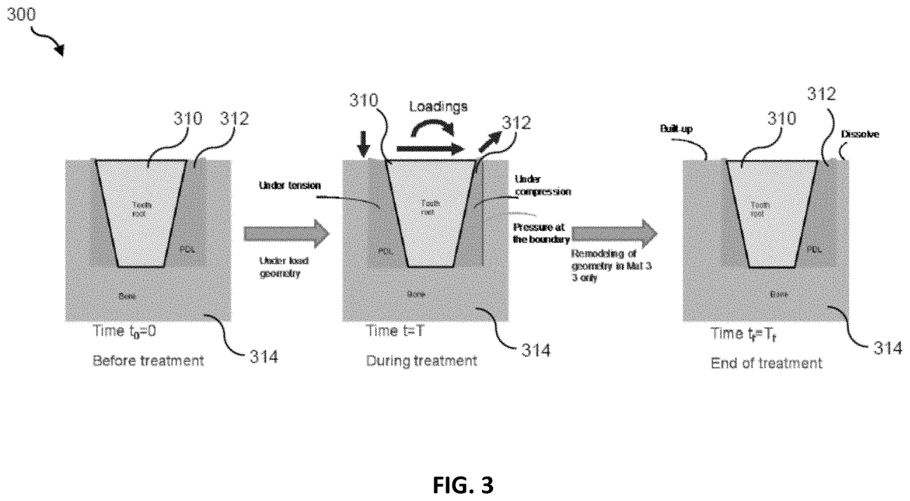

FIG. 3 illustrates a diagram 300 of bone remodeling around a tooth. FIG. 3 shows a tooth root 310 of the tooth, a periodontal ligament (PDL) 312, and a bone 314. At time t0=0 (e.g., before treatment), no force may be applied to tooth root 310. At time T (e.g., during treatment), an appliance may exert various contact forces on the tooth, which may apply various loads to tooth root 310. As seen in FIG. 3, PDL 312 may compress at an interface between tooth root 310 and PDL 312 in a direction of the load and may be under tension. The compression may further create pressure at an interface between PDL 312 and bone 314, which may cause bone 314 to dissolve at this interface. The tension at the opposite interface between PDL 312 and bone 314 may cause bone 314 to build up at this opposite interface. Thus, at time t_{f}=T_{f} (e.g., at an end of treatment), PDL 312 may retain its original shape but may result in bone remodeling for bone 314. Although PDL 312 may retain its shape, the position of tooth 310 has shifted within bone 314.

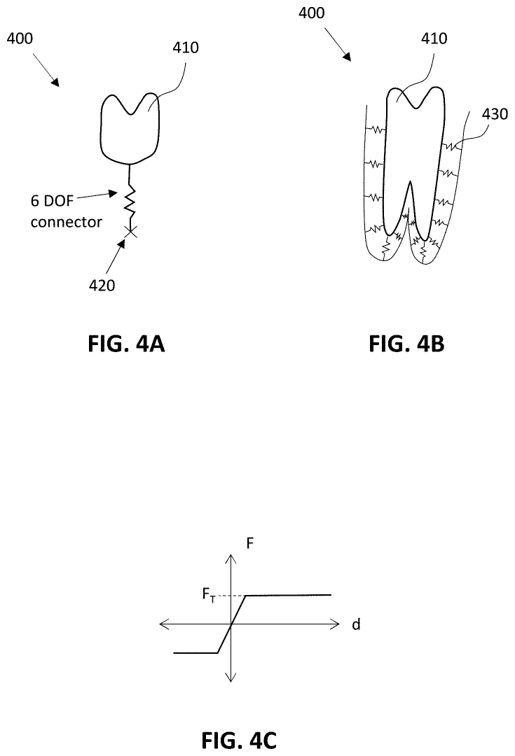

Evaluation module 108 may use model 128 to simulate the above described bone modeling. In one example, model 128 may use single element analysis. More specifically, model 128 may use a 6 degree-of-freedom (DOF) spring to represent a periodontal ligament (PDL) of the examined tooth, which may be anchored to the bone. FIGS. 4A and 4B illustrate diagrams 400 of possible bone remodeling models. A tooth 410 may be connected to a 6 DOF spring 420 that may represent the PDL. FIG. 4C illustrates forces acting on one of the springs.

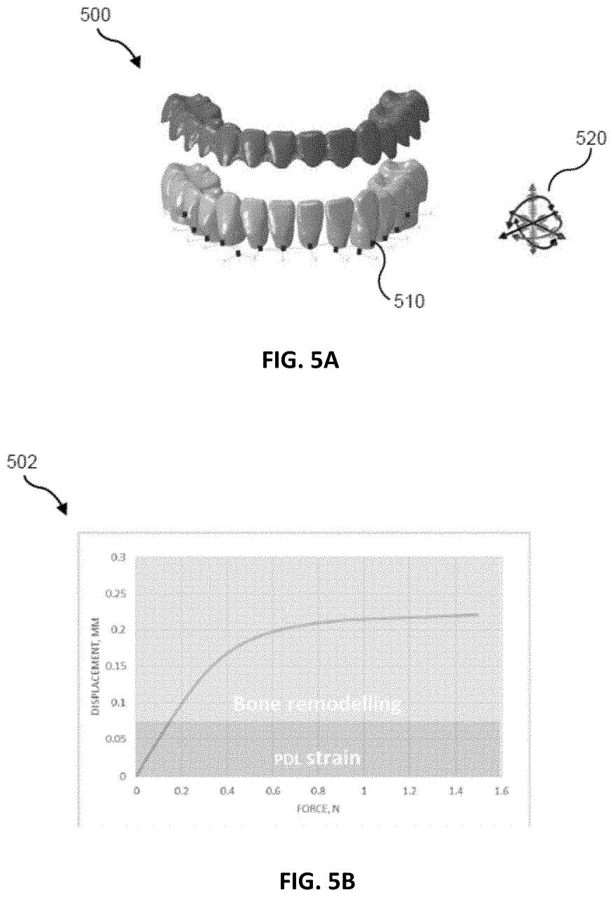

The bone remodeling may be simulated to occur when the 6 DOF spring is displaced. The 6 DOF spring may be displaced according to whether a force applied to the 6 DOF spring (e.g., due to an appliance having appliance design 126) meets a force criterion. If the force criteria is/are met, the 6 DOF spring may be displaced permanently as if the bone is remodeling. FIG. 5A illustrates a model 500 in which a PDL 510 may be simulated as a 6 DOF spring 520. FIG. 5B illustrates a graph 502 of a possible force criterion.

The force criteria may correspond to a plastic flow rule. Alternatively, the force criteria may correspond to a nonlinear hardening rule. Model 128 may include adjusted spring parameters for the 6 DOF spring based on patient data 122, general dentition data, or data from a clinical database. In addition, model 128 may include adjusted spring parameters for the 6 DOF spring based on a particular tooth type.

In another example, model 128 may use an array of elements. Model 128 may include a first array of springs that may be distributed along a shape of the bone to represent the PDL. The shape of the bone may be determined from patient data 122 or may be based on scan data 132 (e.g., CBCT scan data). Model 128 may also include a second array of springs to represent bone remodeling. FIG. 4B illustrates springs 430 corresponding to the PDL.

The bone remodeling may be based on elastic deformation. For example, model 128 may be calibrated with a hardening curve that may fit clinical studies. In other implementations, the bone remodeling may be based on a non-linear elastic model, such as hyper-elastic models and/or hypo-elastic models.

In yet other examples, model 128 may more fully model the PDL and bone. Model 128 may include a geometry of the bone and a geometry of the PDL as well as that of the tooth roots such that the bone remodeling simulation may calculate stresses at a boundary between the geometry of the bone and the geometry of the PDL. A remodeling function for the bone may depend on the stresses in the PDL region, particularly stresses at the boundaries between the PDL and bone. Model 128 may account for the nonlinear geometry of the PDL and the inhomogeneous distribution of stresses in the PDL to calculate the remodeling function.

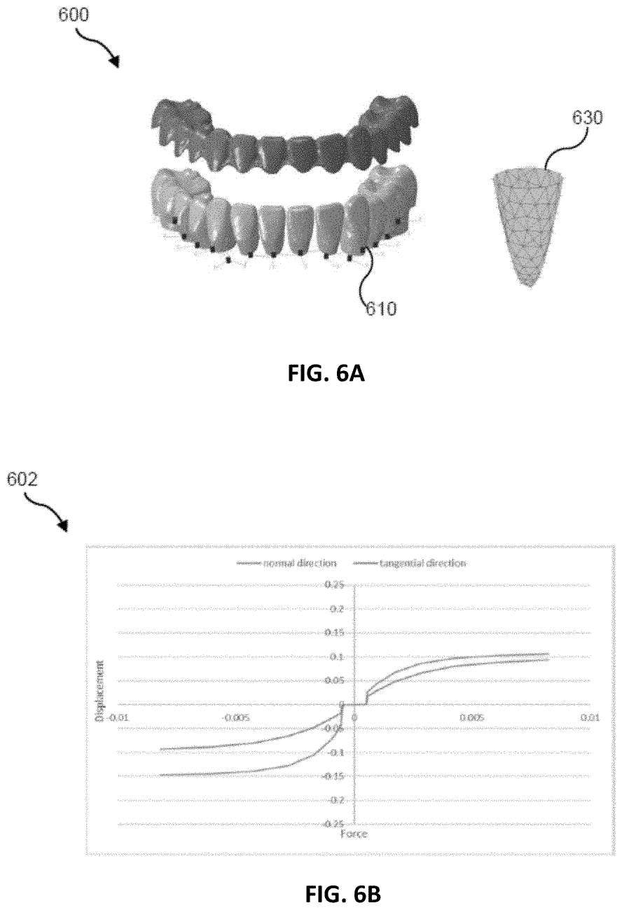

The 3D bone remodeling and PDL deformation may be visualized throughout the simulation. For example, FIG. 6A illustrates how PDL 610 may be modeled with a 3D mesh 630. FIG. 6B illustrates a graph 602 of a remodeling function.

Model 128 may include a solid element that changes a density or a stiffness to represent the bone remodeling. In other implementations, model 128 may include a high viscosity fluid that flows when a strain criterion is satisfied. In yet other implementations, model 128 may include a solid element that changes size based on a stress state of the solid element. In other implementations, model 128 may include a bi-phasic element that shifts phases (e.g., solid to fluid) under a stress state.

Biomechanical modeling may simulate orthodontic tooth movement via precise biomechanical staging based on teeth coronal form, gingiva, teeth roots, alveolar bone, and PDL structure. Although biomechanical modeling may be used for in every case, additional patient data (e.g., scan data 132) may be used to build a full model. Less complex cases may avoid collecting scan data 132 by using contact modeling. Contact modeling, which may be a subset of biomechanical modeling, may simulate efficacy by modeling teeth crowns and using center of resistance estimations to simulate tooth movement. For example, contact modeling may be based on the forces and force moments expected to act on the teeth crowns from an appliance design using the center of resistance estimations. Contact modeling may be used without requiring an exact geometry of teeth roots.

To determine, for example, whether contact modeling may be used, evaluating the appliance design may include evaluating an index of predictability (IoP) for the treatment plan and determining a treatment complexity (e.g., normal, mild, moderate, severe, etc.) based on the IoP. Certain cases may be complex, which may require complex procedures such as mesialization, distalization, arch expansion, teeth extraction, etc. Thus, evaluation module 108 may evaluate treatment plan 124 to determine its complexity. When the complexity is low (e.g., normal, mild, moderate) contact modeling may be used. The complexity may be represented as categories or may be represented numerically such that a complexity threshold may distinguish between low and high complexity.

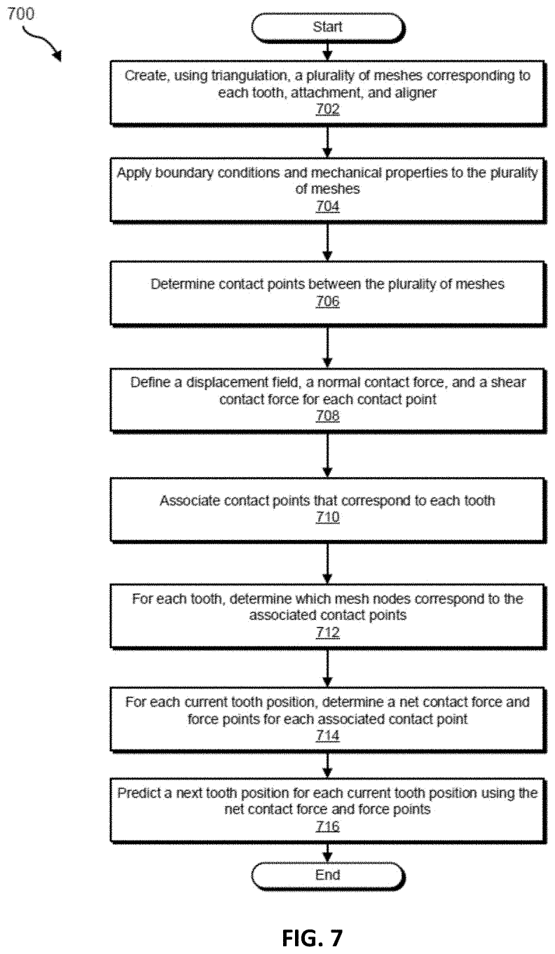

FIG. 7 illustrates an exemplary method 700 for contact modeling. At step 702, one or more of the systems described herein may evaluate the appliance design based on simulation. For example, evaluation module 108 may evaluate appliance design 126 based on a simulation using model 128.

At step 702, one or more of the systems described herein may create, using triangulation, a plurality of meshes corresponding to each tooth, attachment, and aligner. For example, modeling module 110 may create meshes for model 128. At step 704, one or more of the systems described herein may apply boundary conditions and mechanical properties to the plurality of meshes. For example, modeling module 110 may apply boundary conditions and mechanical properties to the meshes of model 128 using finite element modeling (FEM). At step 706, one or more of the systems described herein may determine contact points between the plurality of meshes. For example, modeling module 110 may determine the contact points of model 128 using finite element analysis (FEA).

At step 708, one or more of the systems described herein may define a displacement field, a normal contact force and a shear contact force for each contact point. For example, modeling module 110 may determine the displacement fields, normal contact forces and shear contact forces of model 128. At step 710, one or more of the systems described herein may associate contact points that correspond to each tooth. At step 712, one or more of the systems described herein may evaluate the appliance design based on simulation. For example, modeling module 110 may localize corresponding contact areas for every tooth, and determine which nodes correspond to each contact area.

At step 714, one or more of the systems described herein may, for each current tooth position, determine a net contact force and force points for each associated contact point. For example, modeling module 110 may determine normal and shear components of the net contact forces and force points for each contact area for the current position (e.g., position n).

At step 716, one or more of the systems described herein may predict a next tooth position for each current tooth position using the net contact force and force points. For example, modeling module 110 may use the forces and moments for mechanically-driven staging or optimization of an appliance design to achieve predictable movement of a tooth to a next position (e.g., position n+1).

For high complexity cases, biomechanical modeling may be appropriate. Modeling module 110 may use CBCT data (e.g., scan data 132) for reconstructing 3D shapes of one or more of: teeth, apex positions, teeth root collisions, and a level of orthodontic security to build model 128. Modeling module 110 may use a volumetric neural network, such as a u-net and/or a v-net, for the reconstruction. Modeling module 110 may simulate the efficacy of appliance design 126 using the biomechanical model.

Returning to FIG. 2, at step 208 one or more of the systems described herein may finalize the appliance design based on the evaluation. For example, evaluation module 108 may finalize appliance design 126.

Although method 200 is presented as a sequence of steps, in some examples, the steps of method 200 may be repeated one or more times to redesign and reevaluate appliance design 126 until the simulations indicate sufficient efficacy. Steps 204 and 206 may be repeated until a desired effect (e.g. based on the evaluation at step 206) is achieved. For example, if at step 206 evaluation module 108 determines that appliance design 126 does not satisfy the efficacy threshold (or other success criteria), method 200 may return to step 204 for design module 106 to redesign appliance design 126. In some examples, during the redesign, evaluation module 108 may provide design module 106 with feedback, such as tweaked parameters and/or data, and/or changes to patient data 122 and/or treatment plan 124, for design module 106 use in redesigning appliance design 126. In some examples, design module 106 may prioritize redesigning aspects of appliance design 126 that did not satisfy the efficacy threshold. For example, if an upper appliance of appliance design 126 did not satisfy the efficacy threshold but a lower appliance of appliance design 126 satisfied the efficacy threshold, design module 106 may redesign only the upper appliance. Design module 106 may redesign appliance design 126 using a design scheme similar to that of a previous iteration of step 204. Alternatively, design module 106 may utilize a different scheme to redesign appliance design 126.

Evaluation module 108 may evaluate (e.g., at step 206) redesigned appliance design 126. In some examples, evaluation module 108 may use feedback and/or other data from a previous iteration of step 206. For example, evaluation module 108 may be able to reuse data (e.g., models) from a previous iteration. In some examples, evaluation module 108 may prioritize reevaluating aspects of appliance design 126 that did not satisfy the efficacy threshold in the previous iteration or were otherwise redesigned. For example, appliance design 126 may reevaluate only the upper appliance of appliance design 126 that had previously failed to satisfy the efficacy threshold. The feedback may also indicate changes to data and/or parameters, such as changing (e.g., increasing or decreasing) the efficacy threshold and/or other thresholds.

Evaluation module 108 may evaluate redesigned appliance design 126 using a similar evaluation scheme (e.g., the same type of modeling) as a previous iteration. In some examples, the feedback may indicate changing the evaluation scheme. The change in evaluation scheme may be based in part on the previous failure to satisfy the efficacy threshold. For example, the feedback may indicate increasing a complexity of the case such that modeling module 110 may change from using contact modeling to biomechanical modeling, as described herein. Alternatively, the previous failure to satisfy the efficacy threshold may indicate using a simpler model (e.g., changing from biomechanical modeling to contact modeling). In some examples, the type of modeling as well as other parameters (e.g., efficacy threshold) may be further changed based on other feedback, such as number of failed evaluations, total time elapsed, availability of resources, etc. Moreover, in some examples, step 202 may be repeated. For instance, increasing complexity of the case may require collecting additional patient data 122.

If the reevaluation satisfies the efficacy threshold, method 200 may proceed to step 208 as described herein. Otherwise, one or more steps of method 200, such as steps 204-206, may be reiterated as needed, as described herein. In addition, one or more sub-steps to the steps of method 200 (e.g. the steps of methods 700, 800, and/or 900 as described herein) may be repeated and/or reiterated as needed.

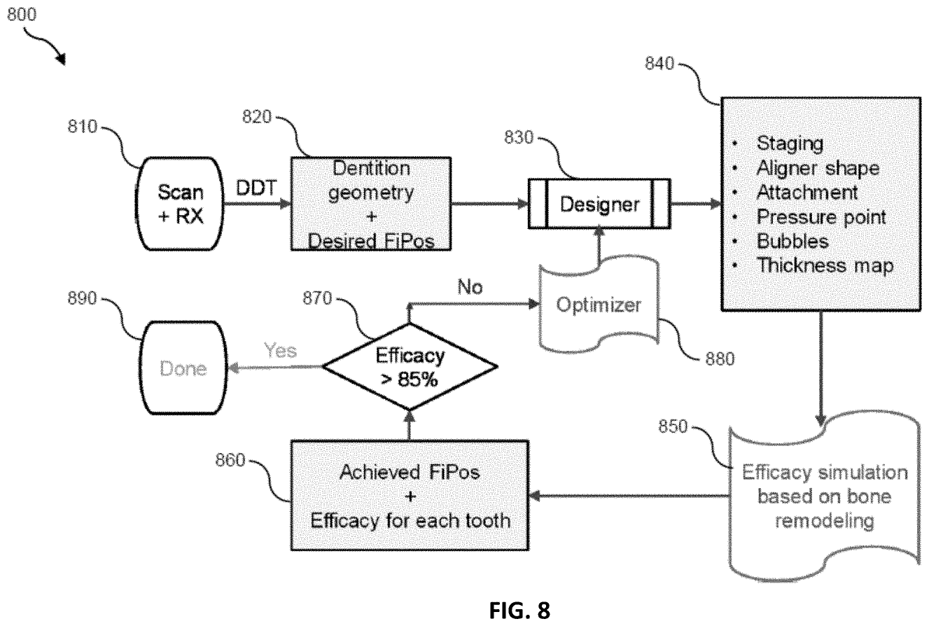

FIG. 8 is a flow diagram of an exemplary computer-implemented method 800 for simulating efficacy. The steps shown in FIG. 8 may be performed by any suitable computer-executable code and/or computing system, including the system illustrated in FIG. 1. In one example, each of the steps shown in FIG. 8 may represent an algorithm whose structure includes and/or is represented by multiple sub-steps, examples of which will be provided in greater detail below. In addition, method 800 may include portions (e.g., steps and/or sub-steps) of method 200 as described herein.

At 810, a patient's treatment planning may begin with scanning the patient, for example to acquire patient data 122. At 820, the patient's dentition geometry (e.g., patient data 122) and desired final position may be determined (e.g., as treatment plan 124). At 830, a designer may design an appropriate appliance (e.g., appliance design 126), including, at 840, staging, aligner shape, attachments, pressure points, bubbles, and a thickness map.

At 850, an efficacy simulation may be performed based on bone remodeling, for example, as discussed above with respect to 206. At 860, the simulated efficacy may be compared against an achieved final position and efficacy for each tooth. At 870, if the efficacy is not greater than 85%, for example, then at 880, an optimizer may optimize a design for a designer by altering forces applied to the tooth, shape and locations of appliances features, or other methods, returning to 830. If at 870 the efficacy is greater than 85%, then at 890, the designing may be done. In some instances, if efficacy is not met, then the treatment plan may be altered, for example, a distance or magnitude of tooth movement or rotation may not be met and therefore, the treatment plan may be revised to use a lesser distance or magnitude of movement or rotation for a particular stage of treatment.

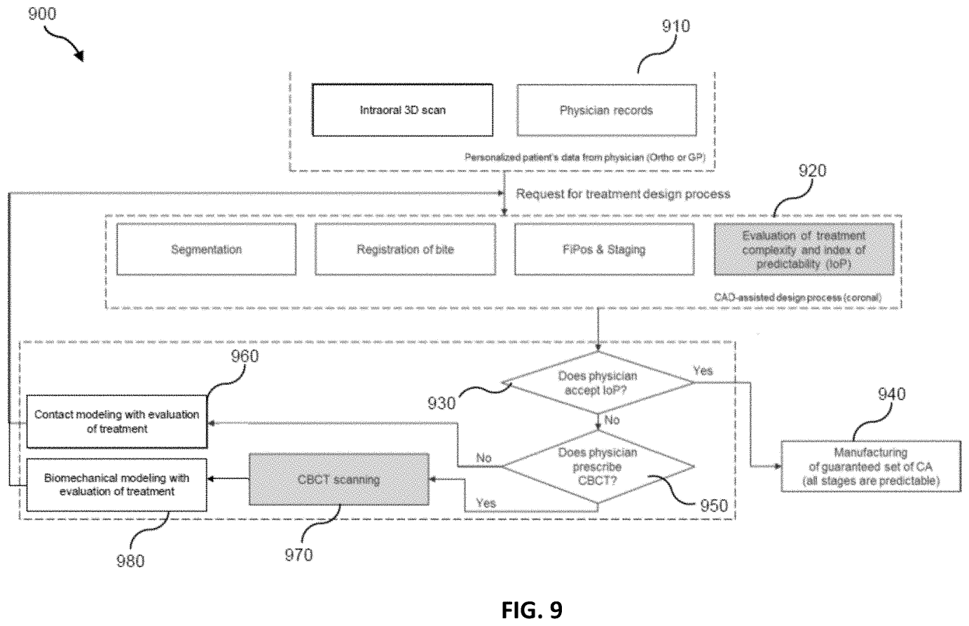

FIG. 9 is a flow diagram of an exemplary computer-implemented method 900 for simulating efficacy. The steps shown in FIG. 9 may be performed by any suitable computer-executable code and/or computing system, including the system illustrated in FIG. 1. In one example, each of the steps shown in FIG. 9 may represent an algorithm whose structure includes and/or is represented by multiple sub-steps, examples of which will be provided in greater detail below. In addition, method 900 may include portions (e.g., steps and/or sub-steps) of method 200 as described herein.

At 910, a patient's treatment planning may begin with personalized patient data, e.g., patient data 122, that may include an intraoral 3D scan and physician records.

At 920, the treatment design process may include segmentation (e.g., identifying various elements from the patient data), registration of bite, final position and staging (e.g., determining stages of treatment to achieve the final position), and an evaluation of treatment complexity and index of predictability (IoP).

At 930, the physician may accept the IoP of the treatment, continuing onto 940 to manufacture appliances for the treatment.

At 950, if the physician did not accept the IoP, the physician may prescribe CBCT scanning, for instance taking into account the treatment complexity.

At 960, if CBCT was not prescribed, contact modeling may be used to evaluate the treatment and the treatment may be redesigned, returning to 920.

At 970, if CBCT was prescribed, the patient may undergo CBCT scanning. At 980, biomechanical modeling may be used to evaluate the treatment and the treatment may be redesigned, returning to 920.

As described above, a patient's treatment may be simulated to determine its efficacy. Various types of simulations may be available, such as bone remodeling, contact modeling, and biomechanical modeling. The treatment and/or associated appliance design may be improved and reevaluated until a threshold efficacy is achieved. Thus, an effective treatment plan may be designed and customized for each patient.

Although the examples herein are described with respect to orthodontic care, in other implementations the remote care may include any other medical care that may require design and fabrication of a custom appliance for treatment.

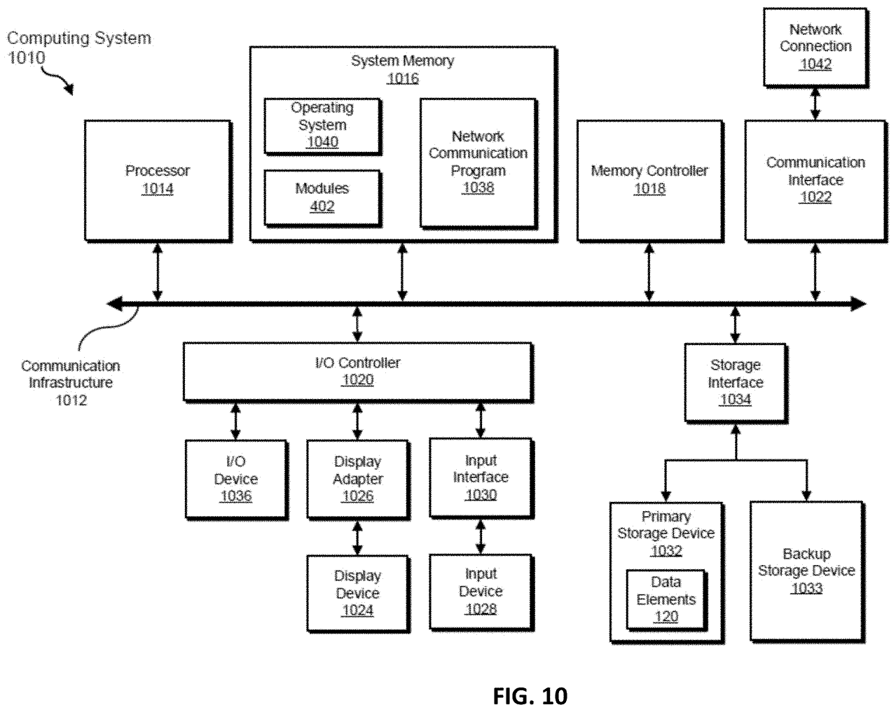

FIG. 10 is a block diagram of an example computing system 1010 capable of implementing one or more of the examples described and/or illustrated herein. For example, all or a portion of computing system 1010 may perform and/or be a means for performing, either alone or in combination with other elements, one or more of the steps described herein (such as one or more of the steps illustrated in FIGS. 2, 7, 8, and 9). All or a portion of computing system 1010 may also perform and/or be a means for performing any other steps, methods, or processes described and/or illustrated herein.

Computing system 1010 broadly represents any single or multi-processor computing device or system capable of executing computer-readable instructions. Examples of computing system 1010 include, without limitation, workstations, laptops, client-side terminals, servers, distributed computing systems, handheld devices, or any other computing system or device. In its most basic configuration, computing system 1010 may include at least one processor 1014 and a system memory 1016.

Processor 1014 generally represents any type or form of physical processing unit (e.g., a hardware-implemented central processing unit) capable of processing data or interpreting and executing instructions. In certain examples, processor 1014 may receive instructions from a software application or module. These instructions may cause processor 1014 to perform the functions of one or more of the example examples described and/or illustrated herein.

System memory 1016 generally represents any type or form of volatile or non-volatile storage device or medium capable of storing data and/or other computer-readable instructions. Examples of system memory 1016 include, without limitation, Random Access Memory (RAM), Read Only Memory (ROM), flash memory, or any other suitable memory device. Although not required, in certain examples computing system 1010 may include both a volatile memory unit (such as, for example, system memory 1016) and a non-volatile storage device (such as, for example, primary storage device 1032, as described in detail below). In one example, one or more of modules 108 from FIG. 1 may be loaded into system memory 1016.

In some examples, system memory 1016 may store and/or load an operating system 1040 for execution by processor 1014. In one example, operating system 1040 may include and/or represent software that manages computer hardware and software resources and/or provides common services to computer programs and/or applications on computing system 1010. Examples of operating system 1040 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain examples, example computing system 1010 may also include one or more components or elements in addition to processor 1014 and system memory 1016. For example, as illustrated in FIG. 10, computing system 1010 may include a memory controller 1018, an Input/Output (I/O) controller 1020, and a communication interface 1022, each of which may be interconnected via a communication infrastructure 1012. Communication infrastructure 1012 generally represents any type or form of infrastructure capable of facilitating communication between one or more components of a computing device. Examples of communication infrastructure 1012 include, without limitation, a communication bus (such as an Industry Standard Architecture (ISA), Peripheral Component Interconnect (PCI), PCI Express (PCIe), or similar bus) and a network.

Memory controller 1018 generally represents any type or form of device capable of handling memory or data or controlling communication between one or more components of computing system 1010. For example, in certain examples memory controller 1018 may control communication between processor 1014, system memory 1016, and I/O controller 1020 via communication infrastructure 1012.

I/O controller 1020 generally represents any type or form of module capable of coordinating and/or controlling the input and output functions of a computing device. For example, in certain examples I/O controller 1020 may control or facilitate transfer of data between one or more elements of computing system 1010, such as processor 1014, system memory 1016, communication interface 1022, display adapter 1026, input interface 1030, and storage interface 1034.

As illustrated in FIG. 10, computing system 1010 may also include at least one display device 1024 coupled to I/O controller 1020 via a display adapter 1026. Display device 1024 generally represents any type or form of device capable of visually displaying information forwarded by display adapter 1026. Similarly, display adapter 1026 generally represents any type or form of device configured to forward graphics, text, and other data from communication infrastructure 1012 (or from a frame buffer, as known in the art) for display on display device 1024.

As illustrated in FIG. 10, example computing system 1010 may also include at least one input device 1028 coupled to I/O controller 1020 via an input interface 1030. Input device 1028 generally represents any type or form of input device capable of providing input, either computer or human generated, to example computing system 1010. Examples of input device 1028 include, without limitation, a keyboard, a pointing device, a speech recognition device, variations or combinations of one or more of the same, and/or any other input device.

Additionally or alternatively, example computing system 1010 may include additional I/O devices. For example, example computing system 1010 may include I/O device 1036. In this example, I/O device 1036 may include and/or represent a user interface that facilitates human interaction with computing system 1010. Examples of I/O device 1036 include, without limitation, a computer mouse, a keyboard, a monitor, a printer, a modem, a camera, a scanner, a microphone, a touchscreen device, variations or combinations of one or more of the same, and/or any other I/O device.

Communication interface 1022 broadly represents any type or form of communication device or adapter capable of facilitating communication between example computing system 1010 and one or more additional devices. For example, in certain examples communication interface 1022 may facilitate communication between computing system 1010 and a private or public network including additional computing systems. Examples of communication interface 1022 include, without limitation, a wired network interface (such as a network interface card), a wireless network interface (such as a wireless network interface card), a modem, and any other suitable interface. In at least one example, communication interface 1022 may provide a direct connection to a remote server via a direct link to a network, such as the Internet. Examples of the network include, without limitation, an intranet, a Wide Area Network (WAN), a Local Area Network (LAN), a Personal Area Network (PAN), the Internet, Power Line Communications (PLC), a cellular network (e.g., a Global System for Mobile Communications (GSM) network), portions of one or more of the same, variations or combinations of one or more of the same, and/or any other suitable network. Communication interface 1022 may also indirectly provide such a connection through, for example, a local area network (such as an Ethernet network), a personal area network, a telephone or cable network, a cellular telephone connection, a satellite data connection, or any other suitable connection.

In certain examples, communication interface 1022 may also represent a host adapter configured to facilitate communication between computing system 1010 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fibre Channel interface adapters, Ethernet adapters, or the like. Communication interface 1022 may also allow computing system 1010 to engage in distributed or remote computing. For example, communication interface 1022 may receive instructions from a remote device or send instructions to a remote device for execution.

In some examples, system memory 1016 may store and/or load a network communication program 1038 for execution by processor 1014. In one example, network communication program 1038 may include and/or represent software that enables computing system 1010 to establish a network connection 1042 with another computing system (not illustrated in FIG. 10) and/or communicate with the other computing system by way of communication interface 1022. In this example, network communication program 1038 may direct the flow of outgoing traffic that is sent to the other computing system via network connection 1042. Additionally or alternatively, network communication program 1038 may direct the processing of incoming traffic that is received from the other computing system via network connection 1042 in connection with processor 1014.

Although not illustrated in this way in FIG. 10, network communication program 1038 may alternatively be stored and/or loaded in communication interface 1022. For example, network communication program 1038 may include and/or represent at least a portion of software and/or firmware that is executed by a processor and/or Application Specific Integrated Circuit (ASIC) incorporated in communication interface 1022.

As illustrated in FIG. 10, example computing system 1010 may also include a primary storage device 1032 and a backup storage device 1033 coupled to communication infrastructure 1012 via a storage interface 1034. Storage devices 1032 and 1033 generally represent any type or form of storage device or medium capable of storing data and/or other computer-readable instructions. For example, storage devices 1032 and 1033 may be a magnetic disk drive (e.g., a so-called hard drive), a solid state drive, a floppy disk drive, a magnetic tape drive, an optical disk drive, a flash drive, or the like. Storage interface 1034 generally represents any type or form of interface or device for transferring data between storage devices 1032 and 1033 and other components of computing system 1010. In one example, data elements 120 from FIG. 1 may be stored and/or loaded in primary storage device 1032.

In certain examples, storage devices 1032 and 1033 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 1032 and 1033 may also include other similar structures or devices for allowing computer software, data, or other computer-readable instructions to be loaded into computing system 1010. For example, storage devices 1032 and 1033 may be configured to read and write software, data, or other computer-readable information. Storage devices 1032 and 1033 may also be a part of computing system 1010 or may be a separate device accessed through other interface systems.

Many other devices or subsystems may be connected to computing system 1010. Conversely, all of the components and devices illustrated in FIG. 10 need not be present to practice the examples described and/or illustrated herein. The devices and subsystems referenced above may also be interconnected in different ways from that shown in FIG. 10. Computing system 1010 may also employ any number of software, firmware, and/or hardware configurations. For example, one or more of the example examples disclosed herein may be encoded as a computer program (also referred to as computer software, software applications, computer-readable instructions, or computer control logic) on a computer-readable medium. The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 1010. All or a portion of the computer program stored on the computer-readable medium may then be stored in system memory 1016 and/or various portions of storage devices 1032 and 1033. When executed by processor 1014, a computer program loaded into computing system 1010 may cause processor 1014 to perform and/or be a means for performing the functions of one or more of the example examples described and/or illustrated herein. Additionally or alternatively, one or more of the example examples described and/or illustrated herein may be implemented in firmware and/or hardware. For example, computing system 1010 may be configured as an Application Specific Integrated Circuit (ASIC) adapted to implement one or more of the example examples disclosed herein.

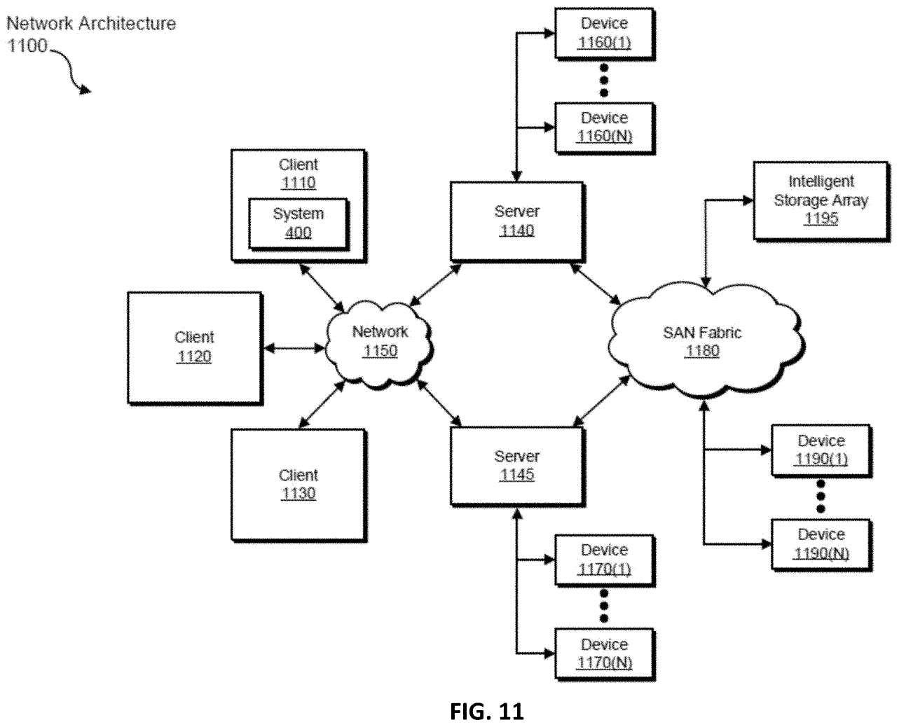

FIG. 11 is a block diagram of an example network architecture 1100 in which client systems 1110, 1120, and 1130 and servers 1140 and 1145 may be coupled to a network 1150. As detailed above, all or a portion of network architecture 1100 may perform and/or be a means for performing, either alone or in combination with other elements, one or more of the steps disclosed herein (such as one or more of the steps illustrated in FIGS. 2, 7, 8, and 9). All or a portion of network architecture 1100 may also be used to perform and/or be a means for performing other steps and features set forth in the instant disclosure.

Client systems 1110, 1120, and 1130 generally represent any type or form of computing device or system, such as example computing system 1010 in FIG. 10. Similarly, servers 1140 and 1145 generally represent computing devices or systems, such as application servers or database servers, configured to provide various database services and/or run certain software applications. Network 1150 generally represents any telecommunication or computer network including, for example, an intranet, a WAN, a LAN, a PAN, or the Internet. In one example, client systems 1110, 1120, and/or 1130 and/or servers 1140 and/or 1145 may include all or a portion of system 100 from FIG. 1.

As illustrated in FIG. 11, one or more storage devices 1160(1)-(N) may be directly attached to server 1140. Similarly, one or more storage devices 1170(1)-(N) may be directly attached to server 1145. Storage devices 1160(1)-(N) and storage devices 1170(1)-(N) generally represent any type or form of storage device or medium capable of storing data and/or other computer-readable instructions. In certain examples, storage devices 1160(1)-(N) and storage devices 1170(1)-(N) may represent Network-Attached Storage (NAS) devices configured to communicate with servers 1140 and 1145 using various protocols, such as Network File System (NFS), Server Message Block (SMB), or Common Internet File System (CIFS).

Servers 1140 and 1145 may also be connected to a Storage Area Network (SAN) fabric 1180. SAN fabric 1180 generally represents any type or form of computer network or architecture capable of facilitating communication between a plurality of storage devices. SAN fabric 1180 may facilitate communication between servers 1140 and 1145 and a plurality of storage devices 1190(1)-(N) and/or an intelligent storage array 1195. SAN fabric 1180 may also facilitate, via network 1150 and servers 1140 and 1145, communication between client systems 1110, 1120, and 1130 and storage devices 1190(1)-(N) and/or intelligent storage array 1195 in such a manner that devices 1190(1)-(N) and array 1195 appear as locally attached devices to client systems 1110, 1120, and 1130. As with storage devices 1160(1)-(N) and storage devices 1170(1)-(N), storage devices 1190(1)-(N) and intelligent storage array 1195 generally represent any type or form of storage device or medium capable of storing data and/or other computer-readable instructions.

In certain examples, and with reference to example computing system 1010 of FIG. 10, a communication interface, such as communication interface 1022 in FIG. 10, may be used to provide connectivity between each client system 1110, 1120, and 1130 and network 1150. Client systems 1110, 1120, and 1130 may be able to access information on server 1140 or 1145 using, for example, a web browser or other client software. Such software may allow client systems 1110, 1120, and 1130 to access data hosted by server 1140, server 1145, storage devices 1160(1)-(N), storage devices 1170(1)-(N), storage devices 1190(1)-(N), or intelligent storage array 1195. Although FIG. 11 depicts the use of a network (such as the Internet) for exchanging data, the examples described and/or illustrated herein are not limited to the Internet or any particular network-based environment.

In at least one example, all or a portion of one or more of the example examples disclosed herein may be encoded as a computer program and loaded onto and executed by server 1140, server 1145, storage devices 1160(1)-(N), storage devices 1170(1)-(N), storage devices 1190(1)-(N), intelligent storage array 1195, or any combination thereof. All or a portion of one or more of the example examples disclosed herein may also be encoded as a computer program, stored in server 1140, run by server 1145, and distributed to client systems 1110, 1120, and 1130 over network 1150.

As detailed above, computing system 1010 and/or one or more components of network architecture 1100 may perform and/or be a means for performing, either alone or in combination with other elements, one or more steps of an example method for virtual care.

While the foregoing disclosure sets forth various examples using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered example in nature since many other architectures can be implemented to achieve the same functionality.

In some examples, all or a portion of example system 100 in FIG. 1 may represent portions of a cloud-computing or network-based environment. Cloud-computing environments may provide various services and applications via the Internet. These cloud-based services (e.g., software as a service, platform as a service, infrastructure as a service, etc.) may be accessible through a web browser or other remote interface. Various functions described herein may be provided through a remote desktop environment or any other cloud-based computing environment.

In various examples, all or a portion of example system 100 in FIG. 1 may facilitate multi-tenancy within a cloud-based computing environment. In other words, the software modules described herein may configure a computing system (e.g., a server) to facilitate multi-tenancy for one or more of the functions described herein. For example, one or more of the software modules described herein may program a server to enable two or more clients (e.g., customers) to share an application that is running on the server. A server programmed in this manner may share an application, operating system, processing system, and/or storage system among multiple customers (i.e., tenants). One or more of the modules described herein may also partition data and/or configuration information of a multi-tenant application for each customer such that one customer cannot access data and/or configuration information of another customer.

According to various examples, all or a portion of example system 100 in FIG. 1 may be implemented within a virtual environment. For example, the modules and/or data described herein may reside and/or execute within a virtual machine. As used herein, the term “virtual machine” generally refers to any operating system environment that is abstracted from computing hardware by a virtual machine manager (e.g., a hypervisor). Additionally or alternatively, the modules and/or data described herein may reside and/or execute within a virtualization layer. As used herein, the term “virtualization layer” generally refers to any data layer and/or application layer that overlays and/or is abstracted from an operating system environment. A virtualization layer may be managed by a software virtualization solution (e.g., a file system filter) that presents the virtualization layer as though it were part of an underlying base operating system. For example, a software virtualization solution may redirect calls that are initially directed to locations within a base file system and/or registry to locations within a virtualization layer.

In some examples, all or a portion of example system 100 in FIG. 1 may represent portions of a mobile computing environment. Mobile computing environments may be implemented by a wide range of mobile computing devices, including mobile phones, tablet computers, e-book readers, personal digital assistants, wearable computing devices (e.g., computing devices with a head-mounted display, smartwatches, etc.), and the like. In some examples, mobile computing environments may have one or more distinct features, including, for example, reliance on battery power, presenting only one foreground application at any given time, remote management features, touchscreen features, location and movement data (e.g., provided by Global Positioning Systems, gyroscopes, accelerometers, etc.), restricted platforms that restrict modifications to system-level configurations and/or that limit the ability of third-party software to inspect the behavior of other applications, controls to restrict the installation of applications (e.g., to only originate from approved application stores), etc. Various functions described herein may be provided for a mobile computing environment and/or may interact with a mobile computing environment.

In addition, all or a portion of example system 100 in FIG. 1 may represent portions of, interact with, consume data produced by, and/or produce data consumed by one or more systems for information management. As used herein, the term “information management” may refer to the protection, organization, and/or storage of data. Examples of systems for information management may include, without limitation, storage systems, backup systems, archival systems, replication systems, high availability systems, data search systems, virtualization systems, and the like.

In some examples, all or a portion of example system 100 in FIG. 1 may represent portions of, produce data protected by, and/or communicate with one or more systems for information security. As used herein, the term “information security” may refer to the control of access to protected data. Examples of systems for information security may include, without limitation, systems providing managed security services, data loss prevention systems, identity authentication systems, access control systems, encryption systems, policy compliance systems, intrusion detection and prevention systems, electronic discovery systems, and the like.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various examples have been described and/or illustrated herein in the context of fully functional computing systems, one or more of these example examples may be distributed as a program product in a variety of forms, regardless of the particular type of computer-readable media used to actually carry out the distribution. The examples disclosed herein may also be implemented using software modules that perform certain tasks. These software modules may include script, batch, or other executable files that may be stored on a computer-readable storage medium or in a computing system. In some examples, these software modules may configure a computing system to perform one or more of the example examples disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some examples one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for designing an appliance for orthodontic treatment, the method comprising:
receiving patient data, and a treatment plan comprising one or more appliances designed for treating a patient;
simulating bone remodeling from the patient data to determine a simulated tooth position using the one or more appliances;
evaluating the one or more appliances by comparing the simulated tooth position to a desired tooth position from the treatment plan; and
modifying the one or more appliances when the comparison between the simulated tooth position and the desired tooth position from the treatment plan exceeds an efficacy threshold.

2. The method of claim 1, wherein the efficacy threshold is 85%.

3. The method of claim 1, wherein simulating bone remodeling includes using a 6 degree-of-freedom (DOF) spring to represent a periodontal ligament (PDL) and the bone remodeling is simulated to occur when the 6 DOF spring is displaced according to a force applied to the 6 DOF spring meeting a force criteria.

4. The method of claim 3, wherein the force criteria corresponds to a plastic flow rule.

5. The method of claim 3, wherein the force criteria corresponds to a nonlinear hardening rule.

6. The method of claim 3, further comprising adjusting spring parameters of the 6 DOF spring based on the patient data.

7. The method of claim 3, further comprising adjusting spring parameters of the 6 DOF spring based on a tooth type corresponding to a periodontal ligament (PDL).

8. The method of claim 1, wherein simulating bone remodeling includes simulating a first array of springs distributed along a shape of bone to represent a periodontal ligament (PDL) and a second array of springs to represent bone remodeling.

9. The method of claim 8, wherein the shape of bone is determined from imaging data.

10. The method of claim 8, wherein the bone remodeling is based on elastic deformation.

11. The method of claim 8, wherein the bone remodeling is based on a non-linear elastic model.

12. The method of claim 1, wherein simulating bone remodeling includes calculating stresses at a boundary between a geometry of bone and a geometry of periodontal ligament (PDL).

13. The method of claim 12, wherein calculating stresses at the boundary comprises simulating one or more of: a solid element that changes a density or a stiffness, a high viscosity fluid that flows when a strain criterion is satisfied, a solid element that changes size based on a stress state of the solid element, and/or a bi-phasic element that shifts phases under a stress state.

14. A method of claim 1, wherein evaluating the one or more appliances further comprises evaluating an index of predictability and determining a treatment complexity based on the index of predictability.

15. The method of claim 14, wherein simulating bone remodeling includes contact modeling when the treatment complexity is low.

16. The method of claim 15, wherein evaluating the one or more appliances further comprises:
creating, using triangulation, a plurality of meshes corresponding to each tooth, attachment, and aligner;
applying boundary conditions and mechanical properties to the plurality of meshes;
determining contact points between the plurality of meshes;
defining a displacement field, a normal contact force, and a shear contact force for each contact point;
associating contact points that correspond to each tooth;
for each tooth, determining which mesh nodes correspond to the contact points;
for each current tooth position, determining a net contact force and force points for each associated contact point; and
predicting a next tooth position for each current tooth position using the net contact force and force points.

17. The method of claim 16, wherein applying boundary conditions and mechanical properties is based on finite element modeling (FEM).

18. The method of claim 16, wherein determining contact points is based on finite element analysis (FEA).

19. The method of claim 14, wherein simulating bone remodeling includes using biomechanical modeling when the treatment complexity is high.

20. The method of claim 19, wherein simulating bone remodeling including using cone beam computed tomography (CBCT) data.

21. The method of claim 19, wherein evaluating the one or more appliances further comprises reconstructing three-dimensional (3D) shapes of at least one of: teeth, apex positions, teeth root collisions, and level of orthodontic security.

22. The method of claim 21, wherein reconstructing utilizes a volumetric neural network.

23. The method of claim 22, wherein the volumetric neural network includes at least one of a u-net and a v-net.

24. A system comprising:
one or more processors; and
a memory coupled to the one or more processors, the memory configured to store computer-program instructions, that, when executed by the one or more processors, perform a computer-implemented method comprising:
receiving patient data, and a treatment plan comprising one or more appliances designed for treating a patient;
simulating bone remodeling from the patient data to determine a simulated tooth position using the one or more appliances;
evaluating the one or more appliances by comparing the simulated tooth position to a desired tooth position from the treatment plan; and
modifying the one or more appliances when the comparison between the simulated tooth position and the desired tooth position from the treatment plan exceeds an efficacy threshold.

25. A non-transitory computer-readable medium comprising one or more computer-executable instructions that, when executed by at least one processor of a computing device, cause the computing device to:
receive patient data, and a treatment plan comprising one or more appliances designed for treating a patient;
simulate bone remodeling from the patient data to determine a simulated tooth position using the one or more appliances;
evaluate the one or more appliances by comparing the simulated tooth position to a desired tooth position from the treatment plan; and
modify the one or more appliances when the comparison between the simulated tooth position and the desired tooth position from the treatment plan exceeds an efficacy threshold.
